(19) United States
(12) Patent Application Publication
(10) Pub. No.: US 2024/0387053 A1
(43) Pub. Date: Nov. 21, 2024

(54) METHOD AND SYSTEM FOR CALIBRATION OF MICROBIOME FOR PERSONALIZED HEALTHCARE

(71) Applicant: Iom Bioworks Private Limited, Bengaluru (IN)

(72) Inventors: Bipin Pradeep Kumar, Bengaluru (IN); Samik Ghosh, Tokyo (JP); Renuka Manchiraju, Bangalore (IN); Nishad Bapatdhar, Bangalore (IN); Sarvesh Menon, Chennai (IN); Dinakaran P, Bangalore (IN)

(21) Appl. No.: 18/664,785

(22) Filed: May 15, 2024

(30) Foreign Application Priority Data
May 15, 2023 (IN) 202341033910

(51) Int. Cl. G16H 50/30; A61B 10/00; G16H 10/40

(52) U.S. Cl. CPC G16H 50/30; A61B 10/0038; G16H 10/40; A61B 2010/0061

(57) ABSTRACT

A wellness measurement method for measuring a level of wellness of an individual based on wellness function using a wellness measurement system is disclosed. The method includes steps of: measuring an approximation of the wellness function for computing the level of wellness of the individual based on microbiome data of the individual; determining wellness areas using symptom classes and corresponding diseases associated with symptoms based on the approximation of the wellness function; determining possible wellness gain using the maximization technique by assigning weightage to the determined wellness areas and a microbiome-wellness association network, and predicting an optimal value of microbiome configuration for the individual based on the wellness areas and the microbiome-wellness association network, being assigned with weightage; and outputting recommendations with the wellness gain through a plurality of constraints using a convex optimization.

### CROSS-REFERENCE TO RELATED APPLICATIONS

This Application claims priority from a patent application filed in India having patent application Ser. No. 202341033910 filed on May 15, 2023.

### FIELD OF INVENTION

Embodiments of the present invention relate to wellness measurement, more particularly relate to a system and method for measuring a level of wellness of an individual based on measuring wellness function by analyzing microbiome data.

### BACKGROUND

A microbiome is an ecological community of commensal, symbiotic, and pathogenic microorganisms that are associated with an organism. The human microbiome includes more microbial cells than human cells, but characterization of the human microbiome is still in nascent stages due to limitations in sample processing techniques, genetic analysis techniques, and resources for processing large amounts of data. Nonetheless, the microbiome is suspected to play at least a partial role in a number of health/disease-related states (e.g., metabolic disorders, diabetes, auto-immune disorders, gastrointestinal disorders, rheumatoid disorders, neurological disorders, and the like).

There are some existing technologies for determining wellness of the individual (i.e., a human) by analyzing an occurrence of microbiomes indicative of symptoms and diseases associated with the symptoms. In an example embodiment, one of the existing prior art US20190211378A1 discloses methods, compositions, and systems for detecting one or more a cognition health issues by characterizing the microbiome of an individual, monitoring such effects, and/or determining, displaying, or promoting a therapy for the cognition health issues.

In another example embodiment, another prior art KR20210028765A discloses a microbiome-based health information providing a system and a method that are capable of predicting and preventing diseases. The method in the prior art includes (a) collecting life pattern information on eating habits, exercise, and sleep; (a) analyzing intestinal microbes of a user through a fecal sample collection kit in which the collected fecal of the user is inserted; and (c) recommending eating habits, exercise, and sleep information according to the correlation between the intestinal microbes and the eating habits, the amount of exercise and the sleep based on analysis results.

In yet another example embodiment, another prior art EP3283652A1 discloses a method for at least one of characterizing, diagnosing, and treating a mental health associated condition in at least a subject. The prior art relates generally to the field of mental health and more specifically to a new and useful method and system for microbiome-derived diagnostics and therapeutics in the field of mental health.

In yet another example embodiment, another prior art US20220290226A1 discloses a deep metagenomic sequencing of more than 1000 individual gut microbiomes, coupled with detailed long-term diet, fasting, and same-meal postprandial cardiometabolic blood markers analyses. In yet another example embodiment, another prior art US20220148737A1 discloses a health and wellness system and more particularly relates to a system and a method for evaluating wellness of one or more users.

Though the above said existing prior art technologies are involved in determining wellness of the individual, the results of determination of the wellness of the individual are based on statistical patterns identified from a population (i.e., cohort) and are not tailored for individuals. Also, the existing prior art technologies are only able to measure the wellness of the individual based on microbiome of the individual or in comparison to other individuals in their cohort, rather than based on features of the individual. Hence, analyzing of microbiome data, which are unique to an individual, and not reporting of the microbiome data of the individual in the population for measuring wellness of the individual, are more challenges for the existing prior art technologies.

Hence, there is a need for an improved system and method for measuring a level of wellness of an individual based on measuring wellness function by analyzing microbiome data, to address the aforementioned problems thereof.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with one embodiment of the disclosure, a wellness measurement method for measuring a level of wellness of an individual based on wellness function using a wellness measurement system is disclosed. The wellness measurement method includes measuring an approximation of the wellness function for computing the level of wellness of the individual based on microbiome data of the individual. The wellness measurement method further includes determining wellness areas using symptom classes and corresponding diseases associated with symptoms based on the approximation of the wellness function.

The wellness measurement method further includes determining wellness gain using the maximization technique by assigning weightage to at least one of: the determined wellness areas and a microbiome-wellness association network, and predicting an optimal value of microbiome configuration for the individual based on at least one of: the wellness areas and the microbiome-wellness association network, being assigned with the weightage. The wellness measurement method further includes outputting personalized recommendations with the wellness gain accurately through a plurality of constraints using a convex optimization.

In an embodiment, measuring the of the approximation of the wellness function comprises (a) assigning a plurality of real variables (i.e., first real variables and the second real variables) to the wellness function, (b) splitting the first real variables into third real variables and fourth real variables based on at least one of: data availability and a user to utilize the first real variables in approximating the wellness function, (c) assigning variables of X, Y, and Z to the third real variables, the fourth real variables, and the second real variables, (d) determining the approximation of the wellness function based on a computation of an approximation to the fourth real variables, and the second real variables at a point of the third real variables, (c) changing the wellness function to be specific to the symptom classes to determine a separate wellness function associated with the symptom classes, and (f) approximating the wellness function for the fourth real variables, and the second real variables for the symptom classes.

The samples of the individual collected from spatial distribution of microbes in an intestine, chemical gradients in a lower intestine, and at least one of: physiological and molecular data, serotonin level of the individual, biological parameters, plasma markers of health conditions comprising a blood glucose level of the individual, and anthropomorphic and phenotypic parameters.

The first real variables comprise microbiome community configuration comprising of: gut microbiome, time, phenotype of the individual, a plurality of types of medical biomarkers comprising physiological and molecular data markers.

A symptom class is an anchor comprising a plurality of symptoms, and the plurality of symptoms are related to each other through corresponding disease classes and mediation strategies.

The approximation of the wellness function for the fourth real variables, and the second real variables is a part of the separate wellness function at the fourth real variables, and the second real variables.

In another embodiment, the first real variables are known real variables, the second real variables are unknown real variables, the third real variables are usable real variables, and the fourth real variables are unusable real variables.

In yet another embodiment, the approximation of the wellness function is measured using at least one of: local point methods, operator spectra technique, a machine learning model (ML), an artificial intelligence (AI) model, stochastic techniques, function class fitting techniques in machine learning and neural nets. The at least one of: local point methods, operator spectra technique, stochastic techniques, function class fitting techniques are selected based on data corresponding to the microbes.

In yet another embodiment, the wellness measurement method further includes approximating the wellness function locally for achieving personalization in a neighborhood of the individual's (102) current state using a wellness mediation strategy.

In yet another embodiment, the wellness areas are determined by combining the separate anchor-specific wellness functions.

In yet another embodiment, the plurality of constraints are added to the convex optimization to restrict a search space to biologically feasible solutions, to perform the convex optimization in a direction for grounding the biologically feasible solutions within a reality of the microbes.

In one aspect, a system for measuring a level of wellness of an individual based on wellness function using a wellness measurement system is disclosed. The system includes a hardware processor and a memory. The memory is coupled to a system bus that couples the memory to the one or more hardware processors. The memory includes a plurality of subsystems stored in the form of executable program which instructs the one or more hardware processors to be executed. The plurality of subsystems includes a wellness function measurement subsystem, a wellness area determining subsystem, a wellness gain determining subsystem, and a recommendation subsystem.

The wellness function measurement subsystem configured to measure an approximation of the wellness function for computing the level of wellness of the individual based on microbiome data of the individual. The wellness area determining subsystem configured to determine wellness areas using symptom classes and corresponding diseases associated with symptoms based on the measured approximation of the wellness function. The wellness gain determination subsystem determines wellness gain to the individual using a maximization technique based on the microbiome data of the individual.

In an embodiment, the wellness gain determination subsystem configured to determine wellness gain using a maximization technique by assigning weightage to at least one of: the determined wellness areas and a microbiome-wellness association network, and predicting an optimal value of microbiome configuration for the individual based on at least one of: the wellness areas and the microbiome-wellness association network being assigned with the weightage. The recommendation subsystem configured to output personalized recommendations with the wellness gain through a plurality of constraints using a convex optimization.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram of a system for measuring a level of wellness of an individual based on wellness function using a wellness measurement system, in accordance with an embodiment of the present disclosure;

FIG. 2 is a block diagram illustrating an exemplary system, such as those shown in FIG. 1, in accordance with an embodiment of the present disclosure;

FIG. 3 is a schematic representation depicting determination of wellness areas using symptom classes and corresponding diseases associated with symptoms, in accordance with an embodiment of the present disclosure;

FIG. 4 is a schematic representation depicting an approximation for anchor specific wellness functions using a complete microbe-anchor network, in accordance with an embodiment of the present disclosure; and

FIG. 5 is a flowchart illustrating a computer implemented method for measuring the level of wellness of the individual based on the wellness function using the wellness measurement system, such as those shown in FIG. 1, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

According to one embodiment, anchors are defined as a collection of symptoms or symptom classes. Anchors and symptom classes are used interchangeably throughout this description.

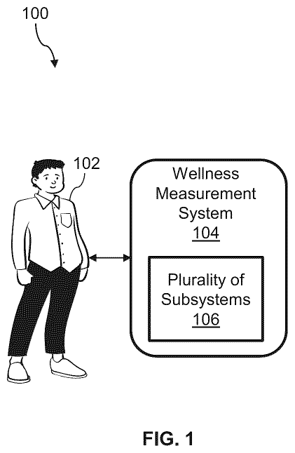

FIG. 1 is a block diagram of a system 100 for measuring a level of wellness of an individual 102 based on wellness function using a wellness measurement system 104, in accordance with an embodiment of the present disclosure. The wellness function refers to a function that uses a plurality of factors and metrics to assess a state of health (i.e., fitness) of the individual 102. In an embodiment, the factors (i.e., variables) include a plurality of types and the plurality of types of the factors is related to various aspects of the individual 102. For example, one such set of factors are associated with a gut microbiome of the individual 102. The system 100 includes a person (i.e., the individual) 102 whose wellness is to be measured, and the wellness measurement system 104. The system 100 measures the level of wellness of the individual 102 based on measuring approximation to the wellness function using microbiome data of the individual 102. The microbiome data are a list of abundances of a plurality of microbes detected in a microbiome of the individual 102. The microbiome data include a list of microbe information in the form of at least one of: genetic information, metabolic information, and the like, from a corresponding sample obtained from the individual 102. In an embodiment, the microbiome data is obtained from a plurality of sources including at least one of: 16S metagenomics, whole metagenomics, and the like. The microbiome data are used with a microbiome-anchor network and a convex optimization to measure a state of wellness of the individual 102. The system computes the state of wellness from the approximation of wellness function based on knowledge of contents of the individual's 102 microbiome. The system 100 further determines wellness areas in the individual 102 based on the symptom classes (i.e., the anchors) and corresponding diseases associated with symptoms in the individual 102.

The system 100 further utilizes a maximization strategy to determine the best possible wellness gain to the individual 102 based on the microbiome data of the individual 102. In an embodiment, the system determines the best possible wellness gain by (a) assigning weightage to the defined wellness areas and a microbiome-wellness association network, and (b) predicting an optimal value of microbiome configuration for the individual 102 based on the wellness areas and the microbiome-wellness association network, being assigned with weightage. The system 100 further approximates the wellness function in a local manner such that the approximation of the wellness function allows for achieving personalization in a neighborhood of the individual's 102 current state.

The system 100 further outputs personalized recommendations through a plurality of constraints that cause predicted data to be generated in a more accurate and biologically meaningful manner. In other words, the system 100 outputs the personalized recommendations with the best possible wellness gain accurately through the plurality of constraints using a convex optimization. The system 100 further measures the maximum wellness of the individual 102, which allows for great flexibility and specificity when deciding from a large amount of data associated with at least one of: wellness areas, the plurality of personalization constraints, feasibility strategies, the microbiome data, and the like.

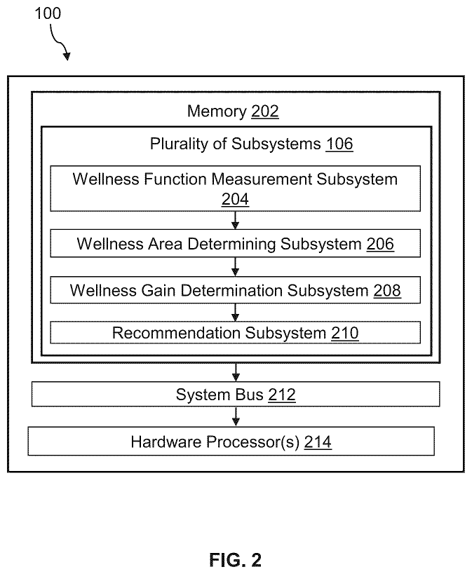

FIG. 2 is a block diagram illustrating an exemplary system 200, such as those shown in FIG. 1, in accordance with an embodiment of the present disclosure. The wellness measurement system 104 includes a hardware processor(s) 214. The wellness measurement system 104 also includes a memory 202 coupled to the hardware processor(s) 214. The memory 202 includes a set of program instructions in the form of the plurality of subsystems 106.

The hardware processor(s) 214, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 202 includes the plurality of subsystems 106 stored in the form of executable program which instructs the hardware processor(s) 214 via a system bus 212 to perform the above-mentioned method steps. The plurality of subsystems 106 include following subsystems: a wellness function measurement subsystem 204, a wellness area determining subsystem 206, a wellness gain determination subsystem 208, and a recommendation subsystem 210.

Computer memory elements may include any suitable memory device(s) for storing data and executable program, such as read only memory, random access memory, erasable programmable read only memory, electronically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 214.

The wellness measurement system 104 includes the wellness function measurement subsystem 204 that is communicatively connected to the hardware processor(s) 214. The wellness function measurement subsystem 204 measures an approximation of the wellness function for computing the level of wellness of the individual 102 by analyzing microbiome data of the individual 102. The wellness function measurement subsystem 204 initially set by considering the wellness function W:D→R over some domain D, where the domain D is defined such that x∈D is a snapshot (i.e., in time and space) of entire contents of a host (i.e., the individual 102). The wellness function measurement subsystem 204 considers W(x) be such that wellness function measurement subsystem 204 measures the abstract notion of wellness of the individual 102, which means the higher W(x), the better the wellness of the host 102. In an embodiment, the wellness function measurement subsystem 204 compares the W(x) to W(x*), for some x and x* in order to measure another unknown wellness of the individual 102. In an embodiment, W(x) is the wellness function at state x, and W(x*) is the wellness function at state x*.

In an embodiment, the wellness function measurement subsystem 204 performs the following operations to measure the approximation of the wellness function for computing the level of wellness of the individual 102 by analyzing microbiome data of the individual 102, wherein the microbiome data of the individual 102 is used as an input to the optimization of the wellness function. The wellness function measurement subsystem 204 initially assigns a plurality of real variables to the wellness function. In an embodiment, the plurality of real variables are extracted from spatial distribution of microbes in an intestine and chemical gradients in a lower intestine and at least one of: physiological and molecular data, an embodiment of which is serotonin level of the individual 102, biological parameters, an embodiment of which is plasma markers of health conditions comprising a blood glucose level of the individual 102, and anthropomorphic and phenotypic parameters. The wellness function (W) is generally highly complex, unknowable and incomputable. The wellness function measurement subsystem 204 measures the approximation of the wellness function (W) to be computable. For example, It is quite possible that W is a wellness function of infinitely many real variables. In an embodiment, the wellness function can technically be defined as a function of finitely many independent variables. In that case,

D=Π_{λϵ[0,1]}{R_{λ}|R_{λ} is the interval of existance of the variable x_{λ}}

In an embodiment, the plurality of real variables may include first real variables and second real variables. In an embodiment, the first real variables are known real variables, and the second real variables are unknown real variables. The unknown real variables are variables that affect the assessment of the wellness state of individuals. The unknown real variables are not currently known or reported in an available knowledge domain. The wellness function measurement subsystem 204 further splits the first real variables (i.e., the known real variables) into third real variables and fourth real variables based on available data and a technical expert to use the known real variables in approximating the wellness function (W). In an embodiment, the third real variables are usable real variables and the fourth real variables are unusable real variables. In an embodiment, the usable real variables are variables that the system 100 has access to measure the approximation of the wellness function (W). For example, the microbiome information at a genus level along with relative abundance data, or the body mass index (BMI) of the individual 102. The unusable real variables are variables that are important in measuring the wellness function. However, the system 100 does not use the unusable variables (e.g., blood chemistry of the individual 102) currently to measure the wellness function. In an embodiment, the known real variables may include microbiome (i.e., bacterial) community configuration including of: gut microbiome, time, phenotype of the individual, a plurality of types of medical biomarkers including physiological and molecular data markers (i.e., serum levels, platelet counts, tissue inflammation level, and the like).

The wellness function measurement subsystem 204 further considers that the wellness function (W) as a function over three sets of variables (X, Y, and Z). Hence, the wellness function measurement subsystem 204 assigns the variables of X, Y, and Z to the usable real variables, the unusable real variables, and the unknown real variables. Further, the wellness function measurement subsystem 204 analyses the domain D (i.e., \tilde{D}) onto the subspace defined by the usable real variables (X). Let \tilde{W}_{Y*,Z*}:\tilde{W}→ [symbol] be defined as

$${\tilde{W}}_{{Y}^{*} , {z}^{*}} ( X ) : W ( X , {Y}^{*} , {Z}^{*} ) , for some fixed ( {Y}^{*} , {Z}^{*} ) \qquad Eqn . ( 1 )$$

In an embodiment, it is clear that \tilde{W}_{Y*,Z*} can be a very poor estimator of the wellness function (W), depending upon how \tilde{W}_{Y*,Z*} fluctuates with choice of (Y*, Z*). In an embodiment, (Y*, Z*) are fixed values for the unusable real variables, and the unknown real variables, as the system 100 is unable to compute the unusable real variables (Y), and the unknown real variables (Z). Hence, the system 100 considers the unusable real variables, and the unknown real variables are the members of the domain D, which are of the form (X, Y, Z*). The wellness function measurement subsystem 204 locally approximates the wellness function (W) to control the strength of such fluctuations. The wellness measurement system 102 accomplishes the approximation of the wellness function to measure the level of wellness of the individual 102 using careful curation of data.

Further, the wellness function measurement subsystem 204 utilizes an indicator (i.e., the real variables that magnify \tilde{W}_{Y*,Z*} fluctuations). The wellness function measurement subsystem 204 uses the indicator to practical use by including the real variables as a bias/parameter. For example, an intestinal wall permeability turns out to be a huge deciding factor for the wellness score of two otherwise identical individuals. In that case, the wellness function measurement subsystem 204 determines the pathology of the phenomenon as well as possibly devising two different approximations of wellness functions \tilde{W} to separately approximate the wellness functions (W) in these two cases.

Since, the real variables are unknowable (Z*) and unusable of the real variables (Y*) at best, the wellness function measurement subsystem 204 measures the approximation to \tilde{W}_{Y*,Z*} at a point X*, which is an approximation to some values lying within a set S_{X*}:={\tilde{W}_{Y,Z}(X*)|(X*, Y, Z)∈D}. The wellness function measurement subsystem 204 changes the wellness function (W) to be specific to the symptom classes (i.e., the anchors). In an embodiment, the changing the definition of the wellness to be specific to the symptom class does not change the original definition of the wellness function. Hence, the wellness function measurement subsystem 204 measures the separate wellness function (W_{Ω}) with respect to the anchor Ω. In an embodiment, the symptom class is otherwise called as the anchor that refers to a collection of symptoms that are related to each other based on the measured wellness function.

The wellness function measurement subsystem 204 approximates the wellness function for the unusable real variables, and the unknown real variables with respect to the symptom classes. In an embodiment, the approximation of the wellness function for the unusable real variables, and the unknown real variables (i.e., \tilde{W}_{Y*,Z*}^{Ω} that is referred to as P_{Ω}), which is a part of the separate wellness function (W_{Ω}) at the unusable real variables, and the unknown real variables (Y*, Z*).

In an embodiment, there are many ways to approximate the wellness function (W) in a meaningful manner using some techniques that relies on available data. A local point method technique to approximate the wellness function (W). The local point methods are smooth enough functions that can be exactly represented by an infinite series. The local point methods may include Taylor expansions having an added property that Taylor residues can be used to exactly measure the error in the approximation when using a truncated series. The fundamental challenge by the local point methods is to determine a biologically meaningful way to define the coefficients for this series, which can be performed by identifying what each term in the series represents.

In another embodiment, an operator spectra technique approximates the wellness function (W). The approximation or modelling the wellness function (W) can be measured indirectly by modeling, using the models, the microbiome using a set of rules and equations. The equations (typically of differential nature) can be framed as operator equations. When the operator includes appropriate properties (e.g., compactness) then the spectrum can be used to practically characterize the wellness function (W).

In an embodiment, the models may include a spring-like network model of the microbiome where edges are likened to springs. A large enough disturbance can cut off an edge. New edges can be formed based on vertex proximity and edges resist stretching. Capturing the requirements of anchor based wellness in the form of a set of equations to be satisfied. This can come from a detailed understanding of the microbiome. The main challenges for the above technique are defining the model, and determining biologically meaningful ways to characterize the wellness function (W). In an embodiment, there are other techniques including at least one of: stochastic techniques to add white noise blankets to mimic real world data collection issues, use of function class fitting techniques (i.e., kernel methods) in ML, neural nets, and the like.

The wellness function measurement subsystem 204 further approximates the wellness function in a local manner for achieving personalization in a neighborhood of the individual's current state using a wellness mediation strategy. The wellness function is a construct that represents an association between biological and physiological features of the individual's 102 body. The wellness function is also an assessment of the state of health or disease exists in the individual 102. Specifically, by considering only the physiological and primary features corresponding to the microbiome, the wellness function is used to measure the state of health or disease of the individual 102. In an embodiment, the function on only some of the physiological and primary features corresponding to the microbiome function, is the approximation to the wellness function. In an exemplary embodiment, the wellness function measurement subsystem 204 considers taxonomy and abundance of a fecal sample of the individual 102 (i.e., X=x₁, x₂, . . . xₙ), where xᵢ is the abundance of the microbiome i (i.e., bacteria i). Further, the current anchors are Sleep, Energy, and Stress (SEnS). Consider that the fecal sample of the individual 102 is given by the vector X *. The wellness function measurement subsystem 204 approximates the wellness function P_{Ω} (where Ω:=SEnS) using its Taylor series, truncated at the linear term.

$${P}_{Ω} ( {X}^{*} + Δ X ) - {P}_{Ω} ( {X}^{*} ) \approx ∇ {P}_{Ω} ( {X}^{*} ) . Δ X \qquad Eqn . ( 2 )$$

The left hand side of the equation (2) is the gain in wellness in moving from X to X*. The wellness function measurement subsystem 204 considers the fecal sample of the individual 102 as an indicator of gut microbiome composition. Further, the right hand side is gradient of P_{Ω} and its dot product in the direction of change. The ∇P_{Ω} is known and extracted from the microbe-disease network. Specifically, the wellness function measurement subsystem 204 approximates ∂P/∂xᵢ (i.e., partial derivative of the wellness function (P) in a direction of the microbe xᵢ) by binary value μᵢ^{Ω}∈{−1, +1} that indicates the nature of the association between microbe i and the anchor Ω. So, in this case,

$$∇ {P}_{Ω} ( {X}^{*} ) . Δ X \approx {\sum }_{i = 1}^{n} {μ}_{i}^{Ω} ( {x}_{i} - {x}_{i}^{*} ) \qquad Eqn . ( 3 )$$

The partial derivative of the wellness function (P) is proportional to the microbe. For example, the partial derivative of the wellness function (P) is positive when the microbe (i.e., xᵢ) increased in the sample is good for the individual 102, as measured by P. The greater the positive wellness change in P with increase in xᵢ, the larger the value of the partial derivative.

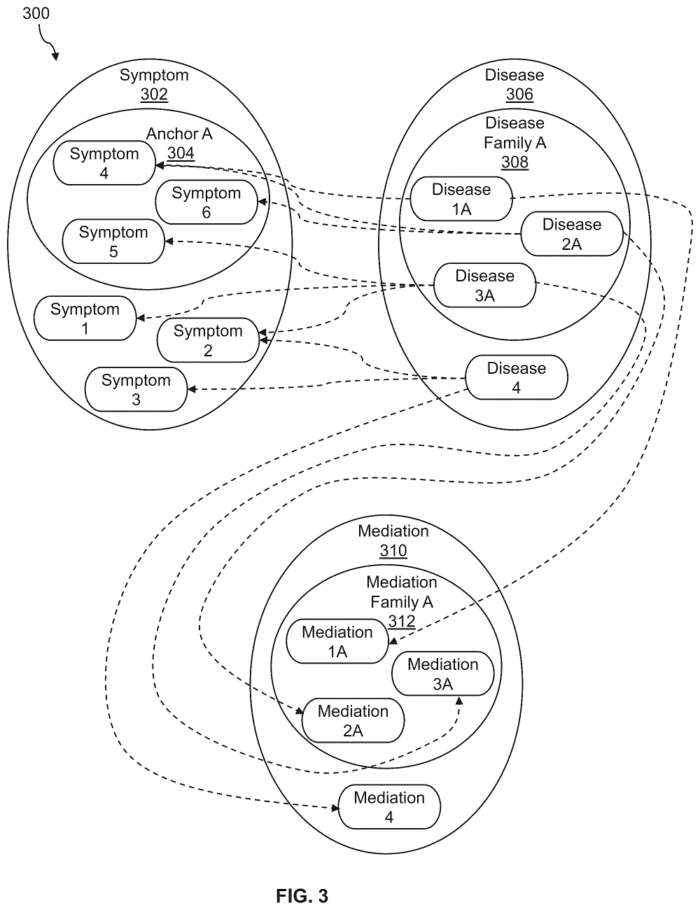

The wellness measurement system 104 includes the wellness area determining subsystem 206 that is communicatively connected to the hardware processor(s) 214. The wellness area determining subsystem 206 determines wellness areas using symptom classes and corresponding diseases associated with symptoms based on the measured approximation of the wellness function. The wellness area determining subsystem 206 aggregates the microbe disease network before its use in optimization. The set of diseases 308 (as shown in FIG. 3) is viewed as a disjoint union of disease families. Each disease family 308 is associated with a family of symptoms (i.e., the symptom class or the anchor) 304. Hence, the microbe-disease association network becomes a microbe-anchor association network such that,

$${μ}_{i}^{Ω} = sign ( {\sum }_{d ∈ Ω} {μ}_{i}^{d} ) \qquad Eqn . ( 4 )$$

where, μᵢ^{d} is the symbolic nature of association of microbe i with disease d.

The above said equations (2) and (3) are approximations to the wellness functions P_{Ω}. The wellness function measurement subsystem 204 is configured to measure the wellness function for a specific anchor using the above said equations (2) and (3) that compute wellness approximations on a subgraph of the microbe-anchor graph including a single anchor and associated microbes connected to it via positive or negative symbolic relationships. Hence, given k anchor specific functions (P_{Ω1}, P_{Ω2}, . . . , P_{ΩK}), the wellness measurement system 102 determines the set of usable real variables (X) in order to simultaneously maximize each of the wellness measurements in the individual 102 (Shown in FIG. 3). Further, the above equation (4) defines the symbolic effect of the microbiome i (i.e., the bacteria i) by symbolic effect over majority of the disease in Ω.

The wellness measurement system 104 includes the wellness gain determination subsystem 208 that is communicatively connected to the hardware processor(s) 214. The wellness gain determination subsystem 208 determines wellness gain (i.e., best possible wellness gain) to the individual 102 using a maximization technique (i.e., the maximization strategy) based on the microbiome data of the individual 102. Based on information of the microbiome data, the microbiome-anchor network is used to form a system of expressions that are to be simultaneously maximized. The maximisation strategy utilizes a convex optimization strategy on a linear combination of the expressions. The system 100 utilizes the microbiome data to provide initial data to the wellness function. For example, when the microbiome includes two bacteria (i.e., bacteria A having initial abundance of 0.3, and bacteria B having initial abundance of 0.6) that are associated positively and negatively with the same anchor, the system 100 maximizes two expressions including A and -B. In an embodiment, A and B are the abundances of the bacteria A and B. Hence, the system 100 creates the linear combination of A-B to maximize the wellness function, subject to a plurality of constraints. The system 100 utilizes the initial abundance of the bacteria as the initial value for the wellness function and maximizes with starting value (0.3-0.6=−0.3). The wellness gain determination subsystem 208 determines the possible wellness gain to the individual using the maximization technique by (a) assigning weightage to the defined wellness areas and the microbiome-wellness association network (i.e., bacteria-wellness association network), and (b) predicting an optimal value of microbiome (i.e., bacterial) configuration for the individual 102 based on the wellness areas and the microbiome-wellness association network, being assigned with weightage. For example, the wellness measurement system 104 initiates with an objective wellness function over the wellness areas. The wellness measurement system 104 further optimizes the wellness function in a local manner using a Taylor approximation. Hence, the wellness measurement system 104 provides the maximum wellness measures for some function of the anchor specific functions, namely, maximization of the function P:R^{k}→R over X.

$$X \to ( {P}_{Ω 1} ( X ) , {P}_{Ω 2} ( X ) , … , {P}_{Ω k} ( X ) ) \to P ( {P}_{Ω 1} , {P}_{Ω 2} , … , {P}_{Ω k} ) \qquad Eqn . ( 5 )$$

This means that,

$$P ( X ) = {\sum }_{i = 1}^{k} {P}_{{Ω}_{i}} ( X )$$

Using Taylor approximation yields,

$$\begin{matrix}P ( {X}^{*} + Δ X ) - P ( {X}^{*} ) & = & ∇ P ( {X}^{*} ) . Δ X & Eqn . ( 6 ) \\ & \approx & {\sum }_{i , j = 1}^{n , k} {μ}_{i}^{{Ω}_{j}} ( {x}_{i} - {x}_{i}^{*} ) & Eqn . ( 7 ) \\ & = & {\sum }_{i = 1}^{n} {μ}_{i} ( {x}_{i} - {x}_{i}^{*} ) & Eqn . ( 8 )\end{matrix}$$

where, X* is the microbiome abundance and taxonomy of the individual 102, and μᵢ:=, Σⱼ₌₁^{k}μᵢ^{Ω}^{ⱼ}.

The wellness gain determination subsystem 208 determines the possible wellness gain to the individual using the maximization technique (i.e., convex optimization). For example, given that the current bacterial abundance for the host microbiome is X*=x*₁, x*₂, x*₃, . . . , x*ₙ, the bacterial abundance that maximises the overall wellness gain for the anchors Ω₁, Ω₂, . . . , Ωₙ is the solution to,

$${max}_{( {x}_{1}^{*} , {x}_{2}^{*} , {x}_{3}^{*} , … , {x}_{n}^{*} )} {\sum }_{i = 1}^{n} {μ}_{i} ( {x}_{i} - {x}_{i}^{*} ) \qquad Eqn . ( 9 a )$$

$$Subject to {x}_{i} >= 0 {∀}_{i} , \qquad Eqn . ( 9 b )$$

$${\sum }_{i = 1}^{n} {x}_{i} = 1 \qquad Eqn . ( 9 c )$$

In an embodiment, the maximization function is a convex optimization (i.e., trivial) and constrained search space is a convex set, which allows the wellness measurement system 104 to use the convex optimization techniques to solve for a global maximum. In an embodiment, the global maximum is the advantage of the convex optimization techniques that allows the wellness measurement system 104 to construct closed form approximations to the target function, so that the wellness measurement system 104 can measure global optimization.

The wellness measurement system 104 further includes the recommendation subsystem 210 that is communicatively connected to the hardware processor(s) 214. The recommendation subsystem 210 outputs personalized recommendations with the best possible wellness gain accurately through a plurality of constraints using a convex optimization technique. In order to drive the optimization in a direction that grounds the solution within the reality of microbiome, the recommendation subsystem 210 uses the same optimization with the plurality of additional constraints being added to restrict the search space to biologically feasible solutions.

In an embodiment, the recommendation subsystem 210 uses the unverified alpha diversity constraint which states: “Alpha diversity of the microbiome is loosely, positively associated with overall wellness”. Thus, the optimisation is constrained to determine the best solution that does not decrease the initial sample alpha diversity, which brings the system 100 to the current formulation of mediation strategy as the following constrained, convex optimisation problem. For example, given that the current bacterial abundance for the host microbiome is X*=x*₁, x*₂, x*₃, . . . , x*ₙ, the bacterial abundance that maximises the overall wellness gain for the anchors Ω₁, Ω₂, . . . , Ωₙ subject to the biological constraints of the microbiome is the solution to,

$${max}_{( {x}_{1} , {x}_{2} , … , {x}_{n} )} {\sum }_{i = 1}^{n} {μ}_{i} ( {x}_{i} - {x}_{i}^{*} ) \qquad Eqn . ( 10 a )$$

$$Subject to {x}_{i} >= 0 {∀}_{i} , \qquad Eqn . ( 10 b )$$

$${\sum }_{i = 1}^{n} {x}_{i} = 1 \qquad Eqn . ( 10 c )$$

$$- {\sum }_{i = 1}^{n} {x}_{i} log ( {x}_{i} ) \geq - {\sum }_{i - 1}^{n} {x}_{i}^{*} log ( {x}_{i}^{*} ) \qquad Eqn . ( 10 d )$$

FIG. 3 is a schematic representation 300 depicting determination of wellness areas using symptom classes (i.e., the anchors) and corresponding diseases associated with symptoms, in accordance with an embodiment of the present disclosure. FIG. 3 shows that the wellness measurement system 104 determines the wellness areas based on the anchors and the corresponding diseases associated with symptoms based on the measured approximation of the wellness function. For example, the disease 1A from the disease family A 308 indicates the symptom 4 in Anchor A 304. Further, the disease 2A from the disease family A 308 indicates the symptom 4 and symptom 6 in Anchor A 304. Similarly, the disease 3A from the disease family A 308 indicates the symptom 5. Symptom 1, and Symptom 2 from the Anchor A 304 and also from outside the Anchor A. Hence, the wellness measurement system 104 simultaneously determines the maximum number of wellness of the individual 102 based on the microbe, disease to microbe and anchor network transformation, as shown in FIG. 3.

FIG. 3 further shows a mediation family A 310 that is a collection of treatments that are common to mediation 1A, mediation 2A, and mediation 3A. A disease (i.e., disease 1A) is associated with a corresponding mediation strategy (i.e., the mediation 1A). FIG. 3 shows a list of all the ways to mitigate and eliminate the diseases from the individual's 102 body in its entirety. The mapping of the disease and the mediation is to indicate that the anchors (i.e., symptom classes) are chosen by analyzing disease classes that cause one or more symptoms of the diseases. Further, the mapping of the disease and the mediation includes commonalities in the mediation strategies, which suggests that a microbiome modulation impacts all diseases in the symptom classes in a similar manner. The wellness measurement system 104 aggregates a disease microbe association in the form of an anchor variable (i.e., μ).

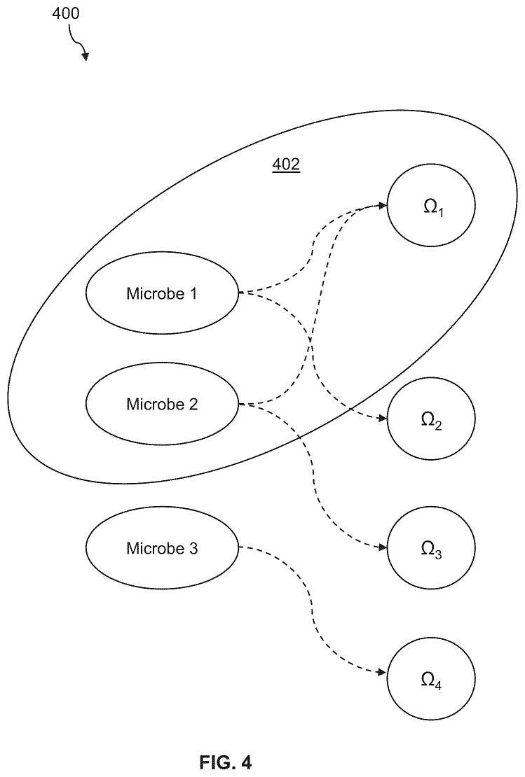

FIG. 4 is a schematic representation 400 depicting an approximation for anchor specific wellness functions using a complete microbe-anchor network, in accordance with an embodiment of the present disclosure. FIG. 4 shows the approximation for the anchor 1 (Ω₁) specific wellness function uses only the subgraph 402 of the complete Microbe (microbe 1 and microbe 2) [symbol] Anchor network, so that the wellness measurement system measures maximum number of wellness of the individual 102 for some function of the anchor specific functions.

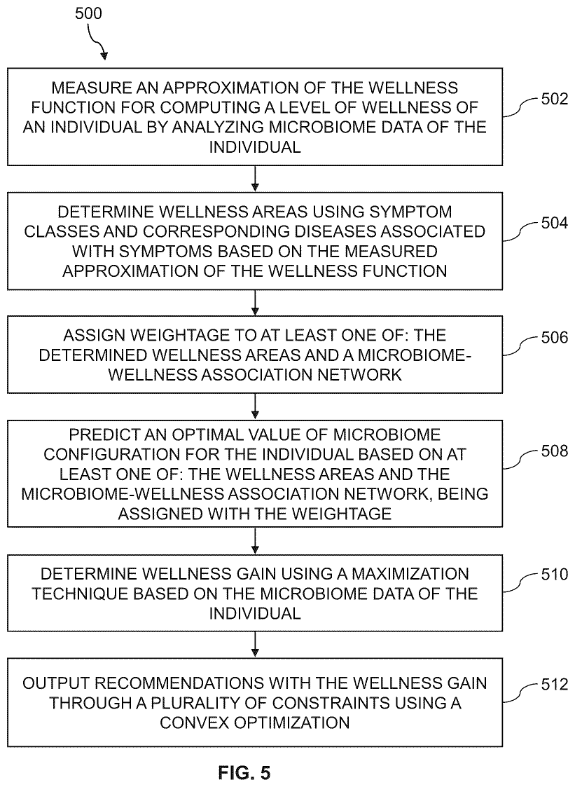

FIG. 5 is a flowchart illustrating a computer implemented method 500 for measuring the level of wellness of the individual based on the wellness function using the wellness measurement system, such as those shown in FIG. 1, in accordance with an embodiment of the present disclosure. At step 502, the approximation of the wellness function is measured for computing the level of wellness of the individual by analyzing microbiome data of the individual 102. At step 504, the wellness areas are determined using the symptom classes (i.e., the anchor) and corresponding diseases associated with symptoms based on the measured approximation of the wellness function. At step 506, the weightage is assigned for at least one of: the determined wellness areas and microbiome-wellness association network.

At step 508, the optimal value of the microbiome configuration for the individual 102 is predicted based on at least one of: the wellness areas and the microbiome-wellness association network being assigned with weightage. At step 510, the best possible wellness gain to the individual 102 using the maximization strategy technique based on the microbiome data of the individual 102. At step 512, personalized recommendations with the best possible wellness gain are outputted accurately through the plurality of constraints using a convex optimization.

The present invention computes the wellness of the individual 102 accurately and in biologically meaningful manner based on the measurements of the wellness function. The present invention further measures the maximum number of the wellness of the individual 102 with some function of the anchor specific functions. The system 100 further determines set of usable real variables (X) such that the system 100 simultaneously maximize each of the wellness measures. The system 100 further measures the maximum wellness of the individual 102, which allows for great flexibility and specificity when deciding from a large amount of data associated with at least one of: wellness areas, the plurality of personalization constraints, feasibility strategies, the microbiome data, and the like.

The present invention further allows an optimization goal to change based on changes in the lifestyle and health conditions of the individuals 102. The present invention further allows changes in temporal dynamics of the individual based on changes in time of the lifestyle and the health conditions of the individuals 102. The present invention further updates the wellness function and the microbiome-wellness area network based on changes in the knowledge for both individuals 102 and population based on knowledge from individual's 102 biology, canonical knowledge of the individual 102, microbiome biology, and population based biological knowledge.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, and the like. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, and the like.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at-least one processor or central processing unit (CPU). The CPUs are interconnected via system bus 212 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, and the like. of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. A wellness measurement method for measuring a level of wellness of an individual based on wellness function using a wellness measurement system, the wellness measurement method comprising:
measuring, by one or more hardware processors, an approximation of the wellness function for computing the level of wellness of the individual based on microbiome data of the individual;
determining, by the one or more hardware processors, wellness areas using symptom classes and corresponding diseases associated with symptoms based on the measured approximation of the wellness function;
determining, by the one or more hardware processors, wellness gain using a maximization technique based on the microbiome data of the individual,
wherein determining the wellness gain using the maximization technique comprises:
assigning, by the one or more hardware processors, weightage to at least one of:
the determined wellness areas and a microbiome-wellness association network; and
predicting, by the one or more hardware processors, an optimal value of microbiome configuration for the individual based on at least one of: the wellness areas and the and microbiome-wellness association network being assigned with the weightage; and
outputting, by the one or more hardware processors, recommendations with the wellness gain through a plurality of constraints using a convex optimization.

2. The wellness measurement method as claimed in claim 1, wherein measuring of the approximation of the wellness function comprises:
assigning, by the one or more hardware processors, a plurality of real variables to the wellness function, wherein the plurality of real variables are extracted from at least one of: samples of the individual, wherein the samples of the individual collected from spatial distribution of microbes in an intestine, chemical gradients in a lower intestine, and at least one of: physiological and molecular data, serotonin level of the individual, biological parameters, plasma markers of health conditions comprising a blood glucose level of the individual, and anthropomorphic and phenotypic parameters, and wherein the plurality of real variables comprise first real variables and second real variables;
splitting, by the one or more hardware processors, the first real variables into third real variables and fourth real variables based on at least one of: data availability and a user to utilize the first real variables in approximating the wellness function, wherein the first real variables comprise microbiome community configuration comprising of: gut microbiome, time, phenotype of the individual, a plurality of types of medical biomarkers comprising physiological and molecular data markers;
assigning, by the one or more hardware processors, variables of X, Y, and Z to the third real variables, the fourth real variables, and the second real variables;
determining, by the one or more hardware processors, the approximation of the wellness function based on a computation of an approximation to the fourth real variables, and the second real variables at a point of the third real variables;
changing, by the one or more hardware processors, the wellness function to be specific to the symptom classes to determine a separate wellness function associated with the symptom classes, wherein a symptom class is an anchor comprising a plurality of symptoms, and wherein the plurality of symptoms are related to each other through corresponding disease classes and mediation strategies; and
approximating, by the one or more hardware processors, the wellness function for the fourth real variables, and the second real variables for the symptom classes, wherein the approximation of the wellness function for the fourth real variables, and the second real variables is a part of the separate wellness function at the fourth real variables, and the second real variables.

3. The wellness measurement method as claimed in claim 2, wherein the first real variables are known real variables, the second real variables are unknown real variables, the third real variables are usable real variables, and the fourth real variables are unusable real variables.

4. The wellness measurement method as claimed in claim 1, wherein the approximation of the wellness function is measured using at least one of: local point methods, operator spectra technique, a machine learning model (ML), an artificial intelligence (AI) model, stochastic techniques, function class fitting techniques in machine learning and neural nets, wherein the at least one of: local point methods, operator spectra technique, stochastic techniques, function class fitting techniques are selected based on data corresponding to the microbes.

5. The wellness measurement method as claimed in claim 1, further comprising approximating, by the one or more hardware processors, the wellness function locally for achieving personalization in a neighborhood of the individual's current state using a wellness mediation strategy.

6. The wellness measurement method as claimed in claim 1, wherein the wellness areas are determined by combining the separate anchor-specific wellness functions.

7. The wellness measurement method as claimed in claim 1, wherein the plurality of constraints are added to the convex optimization to restrict a search space to biologically feasible solutions, to perform the convex optimization in a direction for grounding the biologically feasible solutions within a reality of the microbes.

8. A system for measuring a level of wellness of an individual based on wellness function using a wellness measurement system, the system comprising:
one or more hardware processors; and
a memory operatively coupled to the one or more hardware processors through a system bus, wherein the memory includes a plurality of subsystems stored in the form of executable program which instructs the one or more hardware processors to be executed, wherein the plurality of subsystems comprise:
a wellness function measurement subsystem configured to measure an approximation of the wellness function for computing the level of wellness of the individual based on microbiome data of the individual;
a wellness area determining subsystem configured to determine wellness areas using symptom classes and corresponding diseases associated with symptoms based on the measured approximation of the wellness function;
a wellness gain determining subsystem configured to determine wellness gain using a maximization technique based on the microbiome data of the individual, wherein the wellness gain determining subsystem determines the wellness gain using the maximization technique by
assigning weightage to at least one of: the determined wellness areas and a microbiome-wellness association network; and
predicting an optimal value of microbiome configuration for the individual based on at least one of: the wellness areas and the microbiome-wellness association network being assigned with the weightage; and
a recommendation subsystem configured to output recommendations with the wellness gain through a plurality of constraints using a convex optimization.

9. The system as claimed in claim 8, wherein the wellness function measurement subsystem measures the approximation of the wellness function by
assigning, by the one or more hardware processors, a plurality of real variables to the wellness function, wherein the plurality of real variables are extracted from at least one of: samples of the individual, wherein the samples of the individual collected from spatial distribution of microbes in an intestine, chemical gradients in a lower intestine, and at least one of: physiological and molecular data, serotonin level of the individual, biological parameters, plasma markers of health conditions comprising a blood glucose level of the individual, and anthropomorphic and phenotypic parameters, and wherein the plurality of real variables comprise first real variables and second real variables;
splitting, by the one or more hardware processors, the first real variables into third real variables and fourth real variables based on at least one of: data availability and user to utilize the first real variables in approximating the wellness function, wherein the first real variables comprise microbiome community configuration comprising of: gut microbiome, time, phenotype of the individual, a plurality of types of medical biomarkers comprising physiological and molecular data markers;
assigning, by the one or more hardware processors, variables of X, Y, and Z to the third real variables, the fourth real variables, and the second real variables;
determining, by the one or more hardware processors, the approximation of the wellness function based on a computation of an approximation to the fourth real variables, and the second real variables at a point of the third real variables;
changing, by the one or more hardware processors, the wellness function to be specific to the symptom classes to determine a separate wellness function associated with the symptom classes, wherein a symptom class is an anchor comprising a plurality of symptoms, and wherein the plurality of symptoms are related to each other through corresponding disease classes and mediation strategies; and
approximating, by the one or more hardware processors, the wellness function for the fourth real variables, and the second real variables for the symptom classes, wherein the approximation of the wellness function for the fourth real variables, and the second real variables is a part of the separate wellness function at the fourth real variables, and the second real variables.

10. The system as claimed in claim 9, wherein the first real variables are known real variables, the second real variables are unknown real variables, the third real variables are usable real variables, and the fourth real variables are unusable real variables.

11. The system as claimed in claim 8, wherein the approximation of the wellness function is measured using at least one of: local point methods, operator spectra technique, a machine learning model (ML), an artificial intelligence (AI) model, stochastic techniques, function class fitting techniques in machine learning and neural nets, wherein the at least one of: local point methods, operator spectra technique, stochastic techniques, function class fitting techniques are selected based on data corresponding to the microbes.

12. The system as claimed in claim 8, wherein the wellness function measurement subsystem approximates the wellness function locally for achieving personalization in a neighborhood of the individual's current state using a wellness mediation strategy.

13. The system as claimed in claim 8, wherein the wellness areas are determined by combining the separate anchor-specific wellness functions.

14. The system as claimed in claim 8, wherein the plurality of constraints are added to the convex optimization to restrict a search space to biologically feasible solutions to perform the convex optimization in a direction for grounding the biologically feasible solutions within a reality of the microbes.
